(19) United States
(12) Patent Application Publication
(10) Pub. No.: US 2026/0035734 A1
(43) Pub. Date: Feb. 5, 2026

(54) RAPID eccDNA DETECTION METHOD BASED ON CRISPR-RCA TECHNOLOGY

(71) Applicant: Shanghai Jiao Tong University, Shanghai (CN)

(72) Inventors: Honglu Zhang, Shanghai (CN); Huan Zhang, Shanghai (CN); Qing Guo, Shanghai (CN)

(21) Appl. No.: 19/298,691

(22) Filed: Aug. 13, 2025

(30) Foreign Application Priority Data
Aug. 13, 2024 (CN) 202411108406.1

(51) Int. Cl. C12Q 1/682; G01N 21/64

(52) U.S. Cl. CPC C12Q 1/682; G01N 21/6428; G01N 2021/6439

(57) ABSTRACT

A rapid eccDNA detection method based on CRISPR-RCA technology is provided, which belongs to the field of biotechnology. The method includes the following steps: targeting eccDNA containing a target sequence by using a CRISPR-Cas9 system, carrying out single-stranded DNA probe-assisted enzyme modification at an incision of a target site, directly initiating rolling circle amplification on the target eccDNA at a constant temperature, then targeting and binding a single-stranded rolling circle amplification product by using a CRISPR-Cas14 system to activate the trans-cleavage activity of Cas14, thereby achieving secondary signal amplification.

### SEQUENCE LISTING

The sequence listing is submitted as a XML file filed via EFS-Web, with a file name of “Sequence_Listing.XML”, a creation date of Aug. 10, 2025, and a size of 31,102 bytes. The sequence Listing filed via EFS-Web is a part of the specification and is incorporated in its entirety by reference herein.

### TECHNICAL FIELD

The present disclosure belongs to the field of biological detection and particularly relates to a rapid eccDNA detection method based on CRISPR-RCA technology.

### BACKGROUND

Extrachromosomal circular DNA (eccDNA) carrying random genomic fragments is widely present in different cancer types. The amplification of eccDNA promotes tumor genetic heterogeneity and accelerates tumor evolution. With the progress of analytical techniques, eccDNA has recently been proven to be a multifunctional molecule with various characteristics. The unique topological structure and genetic characteristics of eccDNA provide new ideas for monitoring, early diagnosis, treatment, and prediction of cancers. In existing studies, the primary method for analyzing eccDNA is circular DNA sequencing. This method enables the acquisition of abundant circular DNA sequence information from tumor tissues or normal tissues, facilitating comparative analysis to reveal tumor pathogenesis and even predict cancer progression. However, the circular DNA sequencing method also has some limitations, such as complex operation, long sequencing cycle, and high cost, making it impossible for rapid and highly targeted diagnosis. In recent years, molecular biology techniques have advanced rapidly in the field of rapid disease diagnostics and in vitro diagnostics. The CRISPR-Cas system has emerged as a powerful tool for both gene editing and molecular detection due to its excellent nucleic acid targeting capability. With advantages of low cost, simplicity, portability, high sensitivity, and high specificity, the CRISPR-Cas system is regarded as a “new-generation molecular diagnostic technology”.

Due to the specific recognition, cis-cleavage, and non-specific trans-cleavage capabilities of the CRISPR-Cas system, the detection of nucleic acid targets such as DNA and RNA, as well as non-nucleic acid targets such as proteins, exosomes, cells, and small molecules, has been achieved, and the CRISPR-Cas system has gradually developed into an independent category of molecular detection. The present disclosure has developed an in vitro detection method for eccDNA. This method takes advantage of the circular nature of eccDNA itself, combined with the specific recognition function of the CRISPR-Cas9 system, to amplify the target circular DNA. Subsequently, by using Cas14a for its high-fidelity binding characteristics and the trans-cleavage activity after binding to the target site, the signal at the target site is amplified and the amplified signal is converted into a detectable fluorescence signal. Therefore, it is expected that cancer diagnosis and prognosis could be achieved through the detection of eccDNA using molecular biology techniques.

Currently, the main method for eccDNA detection and analysis is the circular DNA sequencing method (Circle-seq). This research method presents obvious limitations: (1) Long analysis cycle. After acquiring DNA from cells or blood, the linear DNA needs to be digested with exonuclease before eccDNA amplification, and the digestion requires a period of 1-2 weeks. Coupled with rolling circle amplification, gene library preparation and data analysis, the sample submission and detection cycle can last up to 1.5 to 2 months. (2) High detection cost. Due to the need for long-term experimental procedures and the use of gene sequencing instruments, eccDNA sequencing analysis is expensive. (3) Weak detection specificity. Since the sequencing analysis of eccDNA involves sequencing all the circular DNA present within the cells, the detection specificity is relatively weak. The occurrence of some diseases is only related to one or several gene segments present in eccDNA. By detecting the one or several gene segments, the pathological features can be analyzed. If the gene sequencing method is used to detect such eccDNA every time, a long-cycle and costly sequencing process is required. However, only a very small fraction of the vast amount of sequencing data obtained is utilized, resulting in inefficient resource utilization.

### SUMMARY

The present disclosure is intended to overcome the deficiencies of the existing technology and provides a rapid eccDNA detection method based on CRISPR-RCA technology.

In order to achieve the objective of the present disclosure, we will adopt the following technical solution:

A rapid eccDNA detection method based on CRISPR-RCA technology, without digesting linear DNA in the genome, includes the steps of:
S1. binding Cas9n-sgRNA to a target site of a target double-stranded circular DNA by using CRISPR-RCA technology;
S2. synthesizing a single-stranded DNA probe with a length of 40 nt according to the sequence information of the target site, and binding the 40 nt single-stranded DNA probe to a non-complementary strand of sgRNA;
S3. using the single-stranded DNA probe with a length of 40 nt as a template, extending the non-complementary strand of sgRNA at the target site with Klenow large-fragment enzyme;
S4. starting from the extended region of the non-complementary strand of sgRNA and using a single-stranded DNA probe with a length of 20 nt corresponding to the 40 nt single-stranded DNA probe as a primer, performing a strand displacement reaction on the target double-stranded circular DNA to convert the target double-stranded circular DNA to a single-stranded circular DNA;
S5. adding a rolling circle amplification primer to the reaction system and using Phi29 to perform rolling circle amplification on the single-stranded circular DNA obtained from the strand displacement to obtain a rolling circle amplification product;
S6. binding Cas14a-sgRNA and a reporter probe to a target site of the rolling circle amplification product by using CRISPR-RCA technology to activate the trans-cleavage activity and cleave the reporter probe; and
S7. adjusting the volume of the reaction system and performing fluorescence detection under a fluorescence spectrophotometer.

As a preferred solution of the present disclosure, the specific operational steps of the rapid eccDNA detection method are as follows:
S21. selecting the target site on the target double-stranded circular DNA and synthesizing the sgRNA for Cas9n protein according to the sequence information of the target site;
S22. taking a 1.5 mL Ep tube and setting up a reaction system of 20 μL, adding Cas9n protein and sgRNA each at a final concentration of 50 nM, then adding 2 μL of 10×Cas9n Buffer and incubating the mixture at 37° C. for 20 min to allow the Cas9n protein to bind to the sgRNA;
S23. adding the target double-stranded circular DNA to the reaction system at a final concentration of 5 nM, and incubating the mixture at 37° C. for 30 min to allow the Cas9n-sgRNA to bind to the target site of the target double-stranded circular DNA;
S24. adding 1 μL of the single-stranded DNA probe with a length of 40 nt synthesized according to the sequence information of the target site to the reaction system, enabling a final concentration of the 40 nt single-stranded DNA probe to be 500 nM, incubating the mixture at 37° C. for 20 min to allow the 40 nt single-stranded DNA probe to bind to the non-complementary strand of sgRNA;
S25. adding 2 μL of dNTP, 1 μL of Klenow large-fragment enzyme and a corresponding reaction buffer to the reaction system, and using the 40 nt single-stranded DNA probe as the template to extend the non-complementary strand of sgRNA with the Klenow large-fragment enzyme;
S26. adding 2 μL of protease K to the reaction system and incubating the mixture at 52° C. for 60 min to dissociate the Cas9n-sgRNA from the target site;
S27. incubating the entire reaction system at 95° C. for 10 min to inactivate the protease K;
S28. after cooling the reaction system to room temperature, adding 1 μL of a single-stranded DNA probe with a length of 20 nt corresponding to the 40 nt single-stranded DNA probe, enabling a final concentration of the 20 nt single-stranded DNA probe to be 500 nM, then adding 1 μL of Phi29 DNA polymerase and a corresponding reaction buffer to initiate rolling circle amplification which proceeds at 30° C. for 48 h to obtain the rolling circle amplification product;
S29. selecting the target site on the rolling circle amplification product and synthesizing the sgRNA for Cas14a protein according to the sequence information of the target site;
S210. taking a 1.5 mL Ep tube and setting up a reaction system of 40 μL, adding Cas14a protein at a final concentration of 50 nM and sgRNA at a final concentration of 100 nM, then adding 4 μL of 10×Cas14a Buffer and incubating the mixture at 37° C. for 20 min to allow the Cas14a protein to bind to the sgRNA;
S211. adding 20 μL of the rolling circle amplification product to the reaction system, then adding 2 μL of the reporter probe at a final concentration of reporter probe of 2 μM, incubating the mixture at 37° C. for 30 min to allow the Cas14a-sgRNA to bind to the target site on the rolling circle amplification product, thereby activating the trans-cleavage activity and cleaving the reporter probe; and
S212. adjusting the reaction system to a volume of 100 μL and performing fluorescence detection under a fluorescence spectrophotometer.

As a preferred solution of the present disclosure, the incubation time in step S211 is adjusted according to the concentration of the rolling circle amplification product to be detected, and when the concentration of the rolling circle amplification product to be detected is low, the incubation time is extended to 4 h.

As a preferred solution of the present disclosure, the 40 nt single-stranded DNA probe in step S24 binds to the non-complementary strand of sgRNA through complementary base pairing.

As a preferred solution of the present disclosure, the detection sensitivity is improved by increasing the density of Cas14a bound to the rolling circle amplification product.

As a preferred solution of the present disclosure, the sequence of the 20 nt single-stranded DNA probe is: 5′-CTAAG GATGC GTGTA ATTGC-3′ (SEQ ID NO: 1).

As a preferred solution of the present disclosure, the sequence of the reporter probe is:

| (SEQ ID NO: 2) | |
|---|---|
| | FAM-TTTTTTTTTTTT-BHQ1. |

As a preferred solution of the present disclosure, the sequence of the 40 nt single-stranded DNA probe is: 5′-CTAAG GATGC GTGTA ATTGC ATCTC TCTCC TTCTA GCCTC-3′ (SEQ ID NO: 3).

As a preferred solution of the present disclosure, the target double-stranded circular DNA includes, but is not limited to, plasmid DNA, cell lysate DNA, microbial genomic DNA, animal and plant tissue DNA, etc.

### Beneficial Effects

1. Since the present disclosure targets the amplification of eccDNA containing a specific sequence and does not amplify linear DNA and other eccDNA without the target site, there is no need to pre-digest linear DNA during detection. In addition, as there are no steps of constructing a sequencing library and performing on-machine sequencing, the reaction time is significantly shortened compared to the circular DNA sequencing method, thus improving the efficiency of the entire reaction system, and the detection cycle can be shortened to within 60 hours.
2. The eccDNA detection method described in the present disclosure enables the amplification of the target eccDNA at a constant temperature and has a low dependence on large-scale detection instruments, and the main detection cost lies in the synthesis of sgRNA for the Cas14a and Cas9n proteins. Compared with the circular DNA sequencing method, the detection cost is significantly reduced.
3. The present disclosure achieves precise amplification and detection of the target site through two rounds of targeted binding of Cas9n and Cas14a, which overcomes the influence brought by the off-target effect of Cas9n protein, thus enabling the specific detection of eccDNA containing a specific target sequence.
4. The present disclosure, through two signal amplification processes of rolling circle amplification and Cas14a trans-cleavage, and by increasing the density of Cas14a bound on the RCA product to improve the detection sensitivity, achieves a detection limit of 1 fM for double-stranded circular DNA.

### BRIEF DESCRIPTION OF THE DRAWINGS

FIG. 1 is a schematic diagram showing the recognition sites of Cas9n-sgRNA on eccDNA;

FIG. 2 is a schematic diagram showing the rolling circle amplification process mediated by a single-stranded DNA probe;

FIG. 3 is a schematic diagram showing the process of Cas14a binding to the rolling circle amplification product, activating the trans-cleavage activity and cleaving the reporter probe to generate a fluorescence signal;

FIG. 4 shows the detection limit achieved by the system for the circular double-stranded DNA of pUC19;

FIG. 5 shows the detection limit achieved by the present disclosure for the circular double-stranded DNA of PhiX174 under the influence of interfering DNA; and

FIG. 6 shows the detection results of three kinds of eccDNA containing sites related to human hypopharyngeal squamous cell carcinoma by the present disclosure. The experimental groups of eccDNA fragments containing three gene sites showed different degrees of signal enhancement compared with the control group, indicating that the detection of disease-related eccDNA has been achieved.

### DETAILED DESCRIPTION OF THE EMBODIMENTS

The present disclosure will be further described below in conjunction with specific embodiments and drawings.

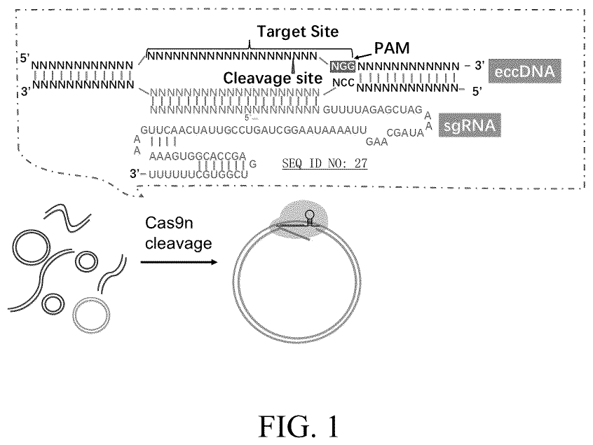

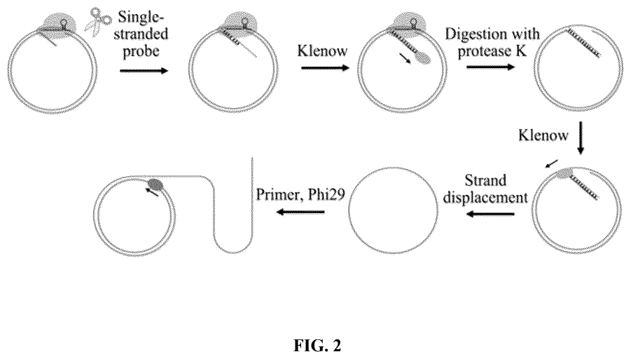

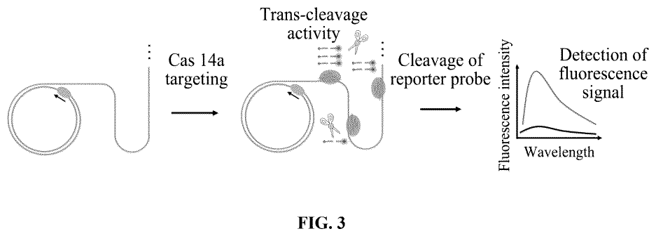

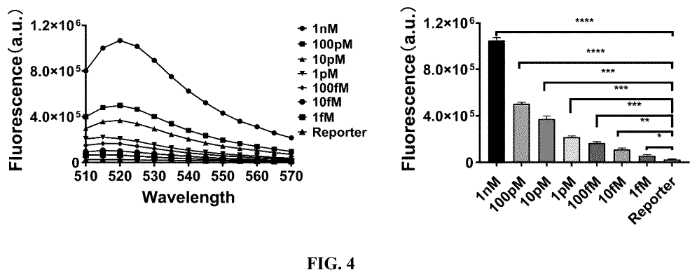

As an embodiment of the present disclosure, as shown in FIGS. 1 to 4, rolling circle amplification was performed on double-stranded circular DNA (pUC19 plasmid) with a length of 2.7 kb using a method mediated by a single-stranded DNA probe.
Step 1: After selecting the target site on the 2.7 kb double-stranded circular DNA (pUC19 plasmid), the sgRNA for Cas9n protein and Cas14a protein were synthesized according to the sequence information of the target site. The sgRNA primers for Cas9n protein and Cas14a protein corresponding to the target site were synthesized by Sangon Biotech and used for the transcription of the sgRNA for Cas9n protein and Cas14a protein corresponding to the target site.

Cas9n-sg-F:
(SEQ ID NO: 4)
5′-TAATA CGACT CACTA TAGGG ACAGA ATCAG GGGAT AACGC
GTTTT AGAGC TAGAA ATAGC AAGTT AAA-3′;
Cas9n-sg-R:
(SEQ ID NO: 5)
5′-GCACC GACTC GGTGC CACTT TTTCA AGTTG ATAAC GGACT
AGCCT TATTT TAACT TGCTA TTTCT AGC-3′;
Cas14a-sg-F:
(SEQ ID NO: 6)
5′-GAAAT TAATA CGACT CACTA TAGGG TTCAC TGATA AAGTG
GAGAA CCGCT TCACC AAAAG CTGTC CCTTA GGGGA TTAGA
ACTTG AGTGA AGGTG GGCTG CTTGC ATCAG CCTAA-3′;
Cas 14a-sg-R:
(SEQ ID NO: 7)
5′-TCACG CTCGT CGTTT GGTAT GTTGC ATTCC TTCAT TCTTT
CAAAT GAATT TGTTT CGAGG GTTAC TTTCC GAAGA AAGCA
CTTCT CGACA TTAGG CTGAT GCAAG CAGCC CACCT-3′.

Step 2: A 1.5 mL Ep tube was used to set up a reaction system of 20 μL. Cas9n protein and the sgRNA corresponding to the target site on the pUC19 double-stranded circular DNA were added, with each component at a final concentration of 50 nM. After then, 2 μL of 10×Cas9n Buffer was added and the mixture was incubated at 37° C. for 20 min to allow the Cas9n protein to bind to the sgRNA.
Step 3: The pUC19 double-stranded circular DNA was added to the reaction system at a final concentration of 5 nM and the mixture was incubated for 30 min to allow the Cas9n-sgRNA to bind to the target site of the target double-stranded circular DNA.
Step 4: 1 μL of the single-stranded DNA probe with a length of 40 nt synthesized according to the sequence of the target site was added to the reaction system such that a final concentration of the 40 nt single-stranded DNA probe was 500 nM, and the mixture was incubated at 37° C. for 20 min to allow the 40 nt single-stranded DNA probe to bind to the non-complementary strand of sgRNA.

The sequence of the 40 nt single-stranded DNA probe: 5′-CTAAG GATGC GTGTA ATTGC GCGTT ATCCC CTGAT TCTGT-3′ (SEQ ID NO: 8).
Step 5: 2 μL of dNTP, 1 μL of Klenow large-fragment enzyme and a corresponding reaction buffer were added to the reaction system, and the 40 nt single-stranded DNA probe was used as the template to extend the non-complementary strand of sgRNA with the Klenow large-fragment enzyme.
Step 6: 2 μL of protease K was added to the reaction system and the mixture was incubated at 52° C. for 60 min to dissociate the Cas9n-sgRNA from the target site.
Step 7: The entire reaction system was incubated at 95° C. for 5 min to inactivate the protease K.
Step 8: After cooling the reaction system to room temperature, 1 μL of 20 nt single-stranded DNA probe (complementary to the black strand in the figure) corresponding to the 40nt single-stranded DNA probe was added such that a final concentration of the 20 nt single-stranded DNA probe was 500 nM. 1 μL of Phi29 DNA polymerase and a corresponding reaction buffer as well as 1 μL of a rolling circle amplification primer were further added such that a final concentration of the rolling circle amplification primer was 500 nM to initiate strand displacement and rolling circle amplification. The rolling circle amplification proceeded at 30° C. for 48 h.

The sequence of the 20 nt single-stranded DNA probe: 5′-CTAAG GATGC GTGTA ATTGC-3′ (SEQ ID NO: 1).

The sequence of the rolling circle amplification primer: GTAAA GCCTG GGGTG CCTAA TG (SEQ ID NO: 9).
Step 9: A 1.5 mL Ep tube was used to set up a reaction system of 40 μL. Cas14a protein was added at a final concentration of 50 nM and a sgRNA corresponding to the PhiX174 double-stranded circular DNA was added at a final concentration of 100 nM. 4 μL of 10×Cas14a Buffer was then added and the mixture was incubated at 37° C. for 20 min to allow the Cas 14a protein to bind to the sgRNA.
Step 10: 20 μL of the rolling circle amplification product obtained in step 7 was added to the reaction system, and 2 μL of the reporter probe (F-Q Reporter) was then added at a final concentration of 2 μM. The mixture was incubated at 37° C. for 30 min to 4 h to allow the Cas14a-sgRNA to bind to the target site on the rolling circle amplification product, thereby activating the trans-cleavage activity and cleaving the reporter probe.

The sequence of the F-Q Reporter: FAM-TTTTTTTTTTTT-BHQ1 (SEQ ID NO: 2).
Step 11: The reaction system was adjusted to a volume of 100 μL and fluorescence detection was performed under a fluorescence spectrophotometer. Where, the fluorescence spectrophotometer was set with an excitation wavelength of 492 nm, a PMT voltage of 650 V, and a scan speed of 240 nm/min.

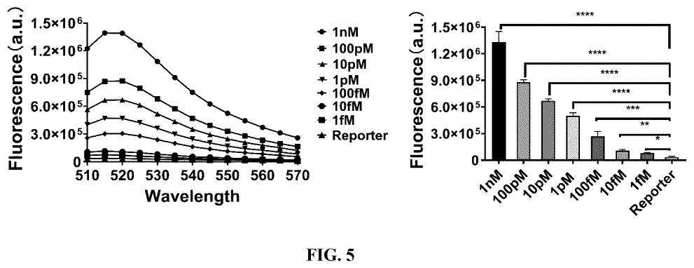

As an embodiment of the present disclosure, as shown in FIGS. 1 to 3 and FIG. 5, different concentrations of PhiX174 were detected using the PhiX174 circular double-stranded DNA with a length of 5.4 kb as the detection object.
Step 1: After selecting the target site according to the sequence information of the PhiX174 double-stranded circular DNA, sgRNA primers for Cas9n protein and Cas14a protein corresponding to the target site were synthesized by Sangon Biotech and used for the transcription of sgRNAs for Cas9n protein and Cas14a protein corresponding to the target site

Cas9n-sg-F:
(SEQ ID NO: 10)
5′-TAATA CGACT CACTA TAGGG AAGGT CATGC GGCAT ACGCT
GTTTT AGAGC TAGAA ATAGC AAGTT AAA-3′;
Cas9n-sg-R:
(SEQ ID NO: 5)
GCACC GACTC GGTGC CACTT TTTCA AGTTG ATAAC GGACT
AGCCT TATTT TAACT TGCTA TTTCT AGC -3′;
Cas14a-sg-F:
(SEQ ID NO: 25)
5′-GAAAT TAATA CGACT CACTA TAGGG TTCAC TGATA AAGTG
GAGAA CCGCT TCACC AAAAG CTGTC CCTTA GGGGA TTAGA
ACTTG AGTGA AG-GTG GGCTG CTTGC ATCAG CCTAA-3′;
Cas14a-sg-R:
(SEQ ID NO: 11)
5′-AACAA TTTAG ACATG GCGCC GTTGC ATTCC TTCAT TCTTT
CAAAT GAATT TGTTT CGAGG GTTAC TTTCC GAAGA AAGCA
CTTCT CGACA TTAGG CTGAT GCAAG CAGCC CACCT-3′.

Step 2: A 1.5 mL Ep tube was used to set up a reaction system of 20 μL. Cas9n protein and the sgRNA corresponding to the target site on the PhiX174 double-stranded circular DNA were added, with each component at a final concentration of 50 nM. After then, 2 μL of 10×Cas9n Buffer was added and the mixture was incubated at 37° C. for 20 min to allow the Cas9n protein to bind to the sgRNA.
Step 3: The PhiX174 double-stranded circular DNA was added to the reaction system at final concentrations ranging from 1 pM to 1 fM. At the same time, M13 double-stranded circular DNA at a concentration of 10 nM and linear PhiX174 double-stranded DNA at a concentration of 1 pM were added to the reaction system and the mixture was incubated at 37° C. for 30 min to allow the Cas9n-sgRNA to bind to the target site of the target double-stranded circular DNA.
Step 4: 1 μL of the single-stranded DNA probe with a length of 40 nt synthesized according to the sequence of the target site was added to the reaction system such that a final concentration of the 40 nt single-stranded DNA probe was 500 nM, and the mixture was incubated at 37° C. for 20 min to allow the 40 nt single-stranded DNA probe to bind to the non-complementary strand of sgRNA.

The sequence of the 40 nt single-stranded DNA probe: 5′-CTAAG GATGC GTGTA ATTGC AGCGT ATGCC GCATG ACCTT-3′_(SEQ ID NO: 12).
Step 5: 2 μL of dNTP, 1 μL of Klenow large-fragment enzyme and a corresponding reaction buffer were added to the reaction system, and the 40 nt single-stranded DNA probe was used as the template to extend the non-complementary strand of sgRNA with the Klenow large-fragment enzyme.
Step 6: 2 μL of protease K was added to the reaction system and the mixture was incubated at 52° C. for 60 min to dissociate the Cas9n-sgRNA from the target site.
Step 7: The entire reaction system was incubated at 95° C. for 5 min to inactivate the protease K.
Step 8: After cooling the reaction system to room temperature, 1 μL of single-stranded DNA probe (complementary to the black strand in the figure) with a length of 20 nt corresponding to the 40 nt single-stranded DNA probe was added such that a final concentration of the 20 nt single-stranded DNA probe was 500 nM. 1 μL of Phi29 DNA polymerase and a corresponding reaction buffer as well as 1 μL of a rolling circle amplification primer were further added such that a final concentration of the rolling circle amplification primer was 500 nM to initiate strand displacement and rolling circle amplification. The rolling circle amplification proceeded at 30° C. for 48 h.

The sequence of the 20 nt single-stranded DNA probe: 5′-CTAAG GATGC GTGTA ATTGC-3′ (SEQ ID NO: 1);

The sequence of the rolling circle amplification primer: CAAAA CGGCA GAAGC CTGAA TG (SEQ ID NO: 13).
Step 9: A 1.5 mL Ep tube was used to set up a reaction system of 40 μL. Cas14a protein was added at a final concentration of 50 nM and a sgRNA corresponding to the PhiX174 double-stranded circular DNA was added at a final concentration of 100 nM. 4 μL of 10×Cas14a Buffer was then added and the mixture was incubated at 37° C. for 20 min to allow the Cas 14a protein to bind to the sgRNA.
Step 10: 20 μL of the rolling circle amplification product obtained in step 7 was added to the reaction system, and 2 μL of the reporter probe (F-Q Reporter) was then added at a final concentration of 2 μM. The mixture was incubated at 37° C. for 30 min to allow the Cas14a-sgRNA to bind to the target site on the rolling circle amplification product, thereby activating the trans-cleavage activity and cleaving the reporter probe.

The sequence of the F-Q Reporter: FAM-TTTTTTTTTTTT-BHQ1 (SEQ ID NO: 2).
Step 11: The reaction system was adjusted to a volume of 100 μL and fluorescence detection was performed under a fluorescence spectrophotometer. Where, the fluorescence spectrophotometer was set with an excitation wavelength of 492 nm, a PMT voltage of 650 V, and a scan speed of 240 nm/min.

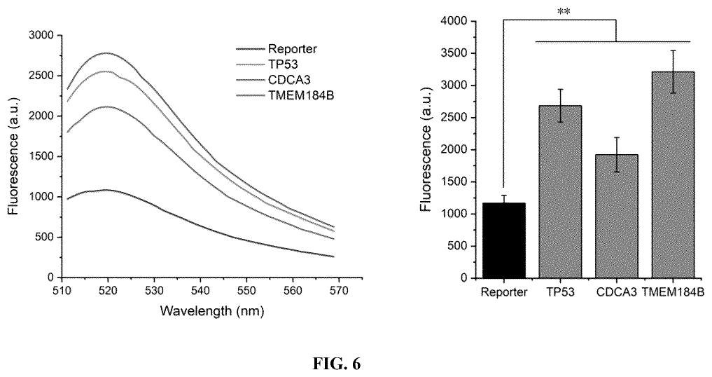

As an embodiment of the present disclosure, as shown in FIGS. 1 to 3 and FIG. 6, eccDNA related to hypopharyngeal squamous cell carcinoma (HSCC) was detected using human hypopharyngeal squamous carcinoma cells (FaDu) as the detection object.
Step 1: Three target gene fragments (TP53, CDCA3, TMEM184B) related to the HSCC disease and present on the eccDNA were screened according to the sequencing information of eccDNA of FaDu cells. According to the sequence information of the gene fragments to be detected on the eccDNA, sgRNA primers for Cas9n protein and Cas14a protein corresponding to the target site on the eccDNA were synthesized by Sangon Biotech and subsequently used for the transcription of sgRNA for Cas9n protein corresponding to the target site.

TP53-Cas9n-sg-F:
(SEQ ID NO: 14)
5′-TAATA CGACT CACTA TAGGG GCGCC ATTGC ACTCT AGCCT
GTTTT AGAGC TAGAA ATAGC AAGTT AAA-3′;
CDCA3-Cas9n-sg-F:
(SEQ ID NO: 15)
5′-TAATA CGACT CACTA TAGGG AGAGC CTGTT CTGCC CCCAG
GTTTT AGAGC TAGAA ATAGC AAGTT AAA-3′;
TMEM184B-Cas9n-sg-F:
(SEQ ID NO: 16)
5′-TAATA CGACT CACTA TAGGG TAGTG ATAGG CAAAG GCCCA
GTTTT AGAGC TAGAA ATAGC AAGTT AAA-3′;
Cas9n-sg-R:
(SEQ ID NO: 5)
GCACC GACTC GGTGC CACTT TTTCA AGTTG ATAAC GGACT
AGCCT TATTT TAACT TGCTA TTTCT AGC-3′;
Cas14a-sg-F:
(SEQ ID NO: 25)
5′-GAAAT TAATA CGACT CACTA TAGGG TTCAC TGATA AAGTG
GAGAA CCGCT TCACC AAAAG CTGTC CCTTA GGGGA TTAGA
ACTTG AGTGA AG-GTG GGCTG CTTGC ATCAG CCTAA-3′;
TP53-Cas14a-sg-R:
(SEQ ID NO: 17)
5′-TAAAG CAGGA GGATA ACTTG GTTGC ATTCC TTCAT TCTTT
CAAAT GAATT TGTTT CGAGG GTTAC TTTCC GAAGA AAGCA
CTTCT CGACA TTAGG CTGAT GCAAG CAGCC CACCT-3′;
CDCA3-Cas14a-sg-R:
(SEQ ID NO: 26)
5′-CAGGG ATACT GAGGA ATGGC GTTGC ATTCC TTCAT TCTTT
CAAAT GAATT TGTTT CGAGG GTTAC TTTCC GAAGA AAGCA
CTTCT CGACA TTAGG CTGAT GCAAG CAGCC CACCT-3′;
TMEM184B-Cas14a-sg-R:
(SEQ ID NO: 18)
5′-GACTT TGCAG AAGTG ACTGA GTTGC ATTCC TTCAT TCTTT
CAAAT GAATT TGTTT CGAGG GTTAC TTTCC GAAGA AAGCA
CTTCT CGACA TTAGG CTGAT GCAAG CAGCC CACCT-3′.

Step 2: A 1.5 mL Ep tube was used to set up a reaction system of 30 μL. Cas9n protein and sgRNAs for Cas9n protein corresponding to three eccDNA target sites were added, with each component at a final concentration of 50 nM. 3 μL of 10×Cas9n Buffer was then added and the mixture was incubated at 37° C. for 20 min to allow the Cas9n protein to bind to the sgRNAs.
Step 3: 10 μg of genomic DNA extracted from FaDu cells was added to the reaction system, which contained all the eccDNA in the cells. The mixture was incubated at 37° C. for 30 min to allow the Cas9n-sgRNA to bind to the target site of the target eccDNA.
Step 4: 1 μL of the single-stranded DNA probe with a length of 40 nt synthesized according to the sequence of the target site was added to the reaction system such that a final concentration of the probe was 500 nM, and the mixture was incubated at 37° C. for 20 min to allow the probe to bind to the non-complementary strand of sgRNA.

The sequence of the 40 nt single-stranded DNA probe at the TP53 site: 5′-CTAAG GATGC GTGTA ATTGC AGGCT AGAGT GCAAT GGCGC-3′ (SEQ ID NO: 19);

The sequence of the 40 nt single-stranded DNA probe at the CDCA3 site: 5′-CTAAG GATGC GTGTA ATTGC CTGGG GGCGA AACAG GCTCT-3′ (SEQ ID NO: 20);

The sequence of the 40 nt single-stranded DNA probe at the TMEM184B site: 5′-CTAAG GATGC GTGTA ATTGC TGGGC CTTTG CCTAT CACTA-3′ (SEQ ID NO: 21).
Step 5: 2 μL of dNTP, 1 μL of Klenow large-fragment enzyme and a corresponding reaction buffer were added to the reaction system, respectively. The 40 nt probe was used as the template to extend the non-complementary strand of sgRNA with the Klenow large- fragment enzyme.
Step 6: 2 μL of protease K was added to the reaction system and the mixture was incubated at 52° C. for 60 min to dissociate the Cas9n-sgRNA from the target site.
Step 7: The entire reaction system was incubated at 95° C. for 5 min to inactivate the protease K.
Step 8: After cooling the reaction system to room temperature, 1 μL of single-stranded DNA probe (complementary to the black strand in the figure) with a length of 20 nt corresponding to the 40 nt single-stranded DNA probe was added such that a final concentration of the 20 nt single-stranded DNA probe was 500 nM. 1 μL of Phi29 DNA polymerase and a corresponding reaction buffer as well as 1 μL of a rolling circle amplification primer were further added such that a final concentration of the rolling circle amplification primer was 500 nM to initiate strand displacement and rolling circle amplification. The rolling circle amplification proceeded at 30° C. for 48 h.

The sequence of the 20 nt single-stranded DNA probe: 5′-CTAAG GATGC GTGTA ATTGC-3′ (SEQ ID NO: 1);

The sequence of the rolling circle amplification primer at the TP53 site: AATCG CTTGA ACCCA GGAGG CA (SEQ ID NO: 22);

The sequence of the rolling circle amplification primer at the CDCA3 site: AAGCG GCCTT CACCC CTAAG TG (SEQ ID NO: 23);

The sequence of the rolling circle amplification primer at the TMEM184B site: CTCTG TCCTT CCCCA GACAA GG (SEQ ID NO: 24).
Step 9: A 1.5 mL Ep tube was used to set up a reaction system of 50 μL. Cas14a protein was added at a final concentration of 50 nM, and sgRNAs for Cas14a protein corresponding to three eccDNA target sites were added with each component at a final concentration of 100 nM. 5 μL of 10×Cas14a Buffer was then added and the mixture was incubated at 37° C. for 20 min to allow the Cas14a protein to bind to the sgRNAs.
Step 10: 30 μL of the rolling circle amplification product obtained in step 7 was added to the reaction system, and 2 μL of the reporter probe (F-Q Reporter) was then added at a final concentration of 2 μM. The mixture was incubated at 37° C. for 30 min to allow the Cas14a-sgRNA to bind to the target site on the rolling circle amplification product, thereby activating the trans-cleavage activity and cleaving the reporter probe.

The sequence of the F-Q Reporter: FAM-TTTTTTTTTTTT-BHQ1 (SEQ ID NO: 2).
Step 11: The reaction system was adjusted to a volume of 100 μL and fluorescence detection was performed under a fluorescence spectrophotometer. Where, the fluorescence spectrophotometer was set with an excitation wavelength of 492 nm, a PMT voltage of 650 V, and a scan speed of 240 nm/min.

The technical solution of the present disclosure has been described in detail above in combination with the embodiments and drawings. However, the present disclosure is not limited to the above technical solution. For those of ordinary skill in the art, several equivalent modifications and substitutions can be made based on the content described herein without departing from the principles of the present disclosure. These equivalent modifications and substitutions shall also be regarded as falling within the scope of protection of the present disclosure.

1. A rapid eccDNA detection method based on CRISPR-RCA technology, the method comprising, without digesting linear DNA in the genome, the steps of:
S1. binding Cas9n-sgRNA to a target site of a target double-stranded circular DNA by using CRISPR-RCA technology;
S2. synthesizing a single-stranded DNA probe with a length of 40 nt according to the sequence information of the target site, and binding the 40 nt single-stranded DNA probe to a non-complementary strand of sgRNA;
S3. using the single-stranded DNA probe with a length of 40 nt as a template, extending the non-complementary strand of sgRNA at the target site with Klenow large-fragment enzyme;
S4. starting from the extended region of the non-complementary strand of sgRNA and using a single-stranded DNA probe with a length of 20 nt corresponding to the 40 nt single-stranded DNA probe as a primer, performing a strand displacement reaction on the target double-stranded circular DNA to convert the target double-stranded circular DNA to a single-stranded circular DNA;
S5. adding a rolling circle amplification primer to the reaction system and using Phi29 to perform rolling circle amplification on the single-stranded circular DNA obtained from the strand displacement to obtain a rolling circle amplification product;
S6. binding Cas14a-sgRNA and a reporter probe to a target site of the rolling circle amplification product by using CRISPR-RCA technology to activate the trans-cleavage activity and cleave the reporter probe; and
S7. adjusting the volume of the reaction system and performing fluorescence detection under a fluorescence spectrophotometer.

2. The rapid eccDNA detection method based on CRISPR-RCA technology according to claim 1, wherein the specific operational steps of the rapid eccDNA detection method are as follows:
S21. selecting the target site on the target double-stranded circular DNA and synthesizing the sgRNA for Cas9n protein according to the sequence information of the target site;
S22. taking a 1.5 mL Ep tube and setting up a reaction system of 20 μL, adding Cas9n protein and sgRNA each at a final concentration of 50 nM, then adding 2 μL of 10×Cas9n Buffer and incubating the mixture at 37° C. for 20 min to allow the Cas9n protein to bind to the sgRNA;
S23. adding the target double-stranded circular DNA to the reaction system at a final concentration of 5 nM, and incubating the mixture at 37° C. for 30 min to allow the Cas9n-sgRNA to bind to the target site of the target double-stranded circular DNA;
S24. adding 1 μL of the single-stranded DNA probe with a length of 40 nt synthesized according to the sequence information of the target site to the reaction system, enabling a final concentration of the 40 nt single-stranded DNA probe to be 500 nM, incubating the mixture at 37° C. for 20 min to allow the single-stranded DNA probe to bind to the non-complementary strand of sgRNA;
S25. adding 2 μL of dNTP, 1 μL of Klenow large-fragment enzyme and a corresponding reaction buffer to the reaction system, and using the 40 nt single-stranded DNA probe as the template to extend the non-complementary strand of sgRNA with the Klenow large-fragment enzyme;
S26. adding 2 μL of protease K to the reaction system and incubating the mixture at 52° C. for 60 min to dissociate the Cas9n-sgRNA from the target site;
S27. incubating the entire reaction system at 95° C. for 10 min to inactivate the protease K;
S28. after cooling the reaction system to room temperature, adding 1 μL of 20 nt probe corresponding to the 40 nt single-stranded DNA probe, enabling a final concentration of the 20 nt single-stranded DNA probe to be 500 nM, then adding 1 μL of Phi29 DNA polymerase and a corresponding reaction buffer to initiate rolling circle amplification which proceeds at 30° C. for 48 h to obtain the rolling circle amplification product;
S29. selecting the target site on the rolling circle amplification product and synthesizing the sgRNA for Cas 14a protein according to the sequence information of the target site;
S210. taking a 1.5 mL Ep tube and setting up a reaction system of 40 μL, adding Cas14a protein at a final concentration of 50 nM and sgRNA at a final concentration of 100 nM, then adding 4 μL of 10×Cas14a Buffer and incubating the mixture at 37° C. for 20 min to allow the Cas14a protein to bind to the sgRNA;
S211. adding 20 μL of the rolling circle amplification product to the reaction system, then adding 2 μL of the reporter probe at a final concentration of reporter probe of 2 μM, incubating the mixture at 37° C. for 30 min to allow the Cas14a-sgRNA to bind to the target site on the rolling circle amplification product, thereby activating the trans-cleavage activity and cleaving the reporter probe; and
S212. adjusting the reaction system to a volume of 100 μL and performing fluorescence detection under a fluorescence spectrophotometer.

3. The rapid eccDNA detection method based on CRISPR-RCA technology according to claim 2, wherein the incubation time in step S211 is adjusted according to the concentration of the rolling circle amplification product to be detected, and when the concentration of the rolling circle amplification product to be detected is low, the incubation time is extended to 4 h.

4. The rapid eccDNA detection method based on CRISPR-RCA technology according to claim 2, wherein the single-stranded DNA probe in step S24 binds to the non-complementary strand of sgRNA through complementary base pairing.

5. The rapid eccDNA detection method based on CRISPR-RCA technology according to claim 1, wherein the detection sensitivity is improved by increasing the density of Cas14a bound to the rolling circle amplification product.

6. The rapid eccDNA detection method based on CRISPR-RCA technology according to claim 1, wherein the sequence of the 20 nt single-stranded DNA probe is: 5′-CTAAG GATGC GTGTA ATTGC-3′ (SEQ ID NO: 1).

7. The rapid eccDNA detection method based on CRISPR-RCA technology according to claim 1, wherein the sequence of the reporter probe is: FAM-TTTTTTTTTTTT-BHQ1 (SEQ ID NO: 2).

8. The rapid eccDNA detection method based on CRISPR-RCA technology according to claim 1, wherein the sequence of the 40 nt single-stranded DNA probe is: 5′-CTAAG GATGC GTGTA ATTGC ATCTC TCTCC TTCTA GCCTC-3′ (SEQ ID NO: 3).

9. The rapid eccDNA detection method based on CRISPR-RCA technology according to claim 1, wherein the target double-stranded circular DNA comprises, but is not limited to, plasmid DNA, cell lysate DNA, microbial genomic DNA, animal and plant tissue DNA, etc.


SEQUENCE LISTING (27 sequences)

SEQ ID NO: 1 - DNA, 20 bp, synthetic construct
  source 1..20: mol_type = other DNA; note = single-stranded DNA probe
ctaaggatgcgtgtaattgc

SEQ ID NO: 2 - DNA, 12 bp, synthetic construct
  source 1..12: mol_type = other DNA; note = reporter probe
tttttttttttt

SEQ ID NO: 3 - DNA, 40 bp, synthetic construct
  source 1..40: mol_type = other DNA; note = single-stranded DNA probe
ctaaggatgcgtgtaattgcatctctctccttctagcctc

SEQ ID NO: 4 - DNA, 68 bp, synthetic construct
  source 1..68: mol_type = other DNA; note = Cas9n-sgRNA primers
taatacgactcactatagggacagaatcaggggataacgcgttttagagctagaaatagcaagttaaa

SEQ ID NO: 5 - DNA, 68 bp, synthetic construct
  source 1..68: mol_type = other DNA; note = Cas9n-sgRNA primers
gcaccgactcggtgccactttttcaagttgataacggactagccttattttaacttgctatttctagc

SEQ ID NO: 6 - DNA, 115 bp, synthetic construct
  source 1..115: mol_type = other DNA; note = Cas14a-sgRNA primers
gaaattaatacgactcactatagggttcactgataaagtggagaaccgcttcaccaaaagctgtcccttaggggattagaacttgagtgaaggtgggctgcttgcatcagcctaa

SEQ ID NO: 7 - DNA, 115 bp, synthetic construct
  source 1..115: mol_type = other DNA; note = Cas14a-sgRNA primers
tcacgctcgtcgtttggtatgttgcattccttcattctttcaaatgaatttgtttcgagggttactttccgaagaaagcacttctcgacattaggctgatgcaagcagcccacct

SEQ ID NO: 8 - DNA, 40 bp, synthetic construct
  source 1..40: mol_type = other DNA; note = single-stranded DNA probe
ctaaggatgcgtgtaattgcgcgttatcccctgattctgt

SEQ ID NO: 9 - DNA, 22 bp, synthetic construct
  source 1..22: mol_type = other DNA; note = rolling circle amplification primer
gtaaagcctggggtgcctaatg

SEQ ID NO: 10 - DNA, 68 bp, synthetic construct
  source 1..68: mol_type = other DNA; note = Cas9n-sgRNA primers
taatacgactcactatagggaaggtcatgcggcatacgctgttttagagctagaaatagcaagttaaa

SEQ ID NO: 11 - DNA, 115 bp, synthetic construct
  source 1..115: mol_type = other DNA; note = Cas14a-sgRNA primers
aacaatttagacatggcgccgttgcattccttcattctttcaaatgaatttgtttcgagggttactttccgaagaaagcacttctcgacattaggctgatgcaagcagcccacct

SEQ ID NO: 12 - DNA, 40 bp, synthetic construct
  source 1..40: mol_type = other DNA; note = single-stranded DNA probe
ctaaggatgcgtgtaattgcagcgtatgccgcatgacctt

SEQ ID NO: 13 - DNA, 22 bp, synthetic construct
  source 1..22: mol_type = other DNA; note = rolling circle amplification primer
caaaacggcagaagcctgaatg

SEQ ID NO: 14 - DNA, 68 bp, synthetic construct
  source 1..68: mol_type = other DNA; note = TP53-Cas9n-sgRNA primers
taatacgactcactataggggcgccattgcactctagcctgttttagagctagaaatagcaagttaaa

SEQ ID NO: 15 - DNA, 68 bp, synthetic construct
  source 1..68: mol_type = other DNA; note = CDCA3-Cas9n-sgRNA primers
taatacgactcactatagggagagcctgttctgcccccaggttttagagctagaaatagcaagttaaa

SEQ ID NO: 16 - DNA, 68 bp, synthetic construct
  source 1..68: mol_type = other DNA; note = TMEM184B-Cas9n-sgRNA primers
taatacgactcactatagggtagtgataggcaaaggcccagttttagagctagaaatagcaagttaaa

SEQ ID NO: 17 - DNA, 115 bp, synthetic construct
  source 1..115: mol_type = other DNA; note = TP53-Cas14a-sgRNA primers
taaagcaggaggataacttggttgcattccttcattctttcaaatgaatttgtttcgagggttactttccgaagaaagcacttctcgacattaggctgatgcaagcagcccacct

SEQ ID NO: 18 - DNA, 115 bp, synthetic construct
  source 1..115: mol_type = other DNA; note = TMEM184B-Cas14a-sgRNA primers
gactttgcagaagtgactgagttgcattccttcattctttcaaatgaatttgtttcgagggttactttccgaagaaagcacttctcgacattaggctgatgcaagcagcccacct

SEQ ID NO: 19 - DNA, 40 bp, synthetic construct
  source 1..40: mol_type = other DNA; note = single-stranded DNA probe
ctaaggatgcgtgtaattgcaggctagagtgcaatggcgc

SEQ ID NO: 20 - DNA, 40 bp, synthetic construct
  source 1..40: mol_type = other DNA; note = single-stranded DNA probe
ctaaggatgcgtgtaattgcctgggggcgaaacaggctct

SEQ ID NO: 21 - DNA, 40 bp, synthetic construct
  source 1..40: mol_type = other DNA; note = single-stranded DNA probe at the TMEM184B site
ctaaggatgcgtgtaattgctgggcctttgcctatcacta

SEQ ID NO: 22 - DNA, 22 bp, synthetic construct
  source 1..22: mol_type = other DNA; note = rolling circle amplification primer at the TP53 site
aatcgcttgaacccaggaggca

SEQ ID NO: 23 - DNA, 22 bp, synthetic construct
  source 1..22: mol_type = other DNA; note = rolling circle amplification primer at the CDCA3 site
aagcggccttcacccctaagtg

SEQ ID NO: 24 - DNA, 22 bp, synthetic construct
  source 1..22: mol_type = other DNA; note = rolling circle amplification primer at the TMEM184B site
ctctgtccttccccagacaagg

SEQ ID NO: 25 - DNA, 115 bp, synthetic construct; identical to SEQ ID NO: 6
  source 1..115: mol_type = other DNA; note = Cas14a-sgRNA primers

SEQ ID NO: 26 - DNA, 115 bp, synthetic construct
  source 1..115: mol_type = other DNA; note = CDCA3-Cas14a-sgRNA primers
cagggatactgaggaatggcgttgcattccttcattctttcaaatgaatttgtttcgagggttactttccgaagaaagcacttctcgacattaggctgatgcaagcagcccacct

SEQ ID NO: 27 - RNA, 82 nt, synthetic construct
  source 1..82: mol_type = other RNA; note = Cas9n-sgRNA
gttttagagctagaaatagcaagttaaaataaggctagtccgttatcaacttgaaaaagtggcaccgagtcggtgctttttt